(19) United States
(12) Patent Application Publication
(10) Pub. No.: US 2025/0268586 A1
(43) Pub. Date: Aug. 28, 2025

(54) SYSTEMS, METHODS, AND DEVICES FOR BIOPSY SAMPLE EXTRACTION

(71) Applicant: APERTURE PUNCH, CORPORATION, Olympia, WA (US)

(72) Inventors: Ashley Lynn WILDHABER, Olympia, WA (US); Nicholas WALTON, Seattle, WA (US); Kenneth Jeffrey PURDY, Seattle, WA (US); Brock PIPER, Chehalis, WA (US)

(73) Assignee: APERTURE PUNCH, CORPORATION, Olympia, WA (US)

(21) Appl. No.: 18/859,146

(22) PCT Filed: Apr. 21, 2023
(86) PCT No.: PCT/US2023/019364

§ 371 Date: Oct. 22, 2024

Related U.S. Application Data
(63) Continuation-in-part of application No. 17/727,078, filed on Apr. 22, 2022.

(51) Int. Cl. A61B 10/02

(52) U.S. Cl. CPC A61B 10/0266; A61B 2010/0208

(57) ABSTRACT

Systems, methods, and devices include a biopsy tissue extraction device for creating, extracting and/or collecting a biopsy tissue sample. The biopsy tissue extraction device includes a housing with an extraction opening at a first distal end and an actuation handle at a second distal end. Extraction blade(s) extend out the extraction opening responsive to an engagement of the actuation handle. The extraction blade(s) move in a linear and/or circular motion, caused by a shaft connecting the extraction blade to the actuation handle, to cut the biopsy sample from a target tissue angle. The extraction blade(s) have a non-perpendicular or rotational approach angle with the target tissue area, such that the blade(s) fully sever the biopsy sample with a single motion.

### CROSS-REFERENCE TO RELATED APPLICATION

This application is a continuation-in-part of and claims priority to U.S. patent application Ser. No. 17/727,078, filed on Apr. 22, 2022 and titled “Systems, Methods, and Devices for Biopsy Sample Extraction,” the entirety of which is incorporated herein by reference.

### BACKGROUND

### 1. Field

The present disclosure relates generally to the field of surgical procedures and, more particularly, to biopsy devices.

### 2. Discussion of Related Art

Creating and retrieving a biopsy sample typically uses multiple tools to make multiple incisions. An initial outline cut of the biopsy sample is created with a biopsy punch, similar to a cookie cutter, which makes a perpendicular incision into the tissue. After creating the outline cut, the biopsy sample tissue is still connected to the surrounding tissue, so the doctor uses a scalpel or forceps to sever the biopsy sample tissue at the base.

This multi-step process can be dangerous and generally requires multiple, trained medical personnel (e.g., a doctor and a nurse) to perform the steps quickly and with enough hands to manage the different tools and the collected biopsy sample. Sometimes severing the biopsy sample at the base requires multiple cuts with the scalpel. A prolonged healing period with significant scarring often occurs because these hand-made cuts are irregular. Moreover, the accuracy of the cuts can be inconsistent such that additional corrective cuts are needed, prolonging the biopsy extraction procedure and causing additional healthy tissue to be removed. Many physicians add an extra 5 mm buffer to the initial outline cut to ensure that a single biopsy procedure sufficiently removes the tissue the biopsy procedure is intended to collect. However, these biopsy samples are significantly larger than necessary—as are the corresponding scars/blemishes on the patients.

Handling the biopsy sample by multiple people often changes/damages the biopsy sample prior to the lab analysis of the biopsy sample. In situations involving contagious tissue, doctors and nurses are exposed to another safety hazard of being infected, in addition to the safety hazard already present by handling multiple exposed blades. Furthermore, lab analysis techniques performed on biopsy samples cannot reliably recreate orientation information (e.g., to determine a top, bottom, left side, or right side) when inconsistent biopsy samples are created using scalpels and forceps.

It is with these observations in mind, among others, that various aspects of the present disclosure were conceived and developed.

### BRIEF SUMMARY

The presently disclosed technology addresses the foregoing problems by providing systems, methods, and devices to extract and collect a biopsy tissue sample. For instance, a biopsy sample extraction device can include an elongated housing with an extraction opening at a first distal end and an actuation handle at a second distal end. Furthermore, the biopsy sample extraction device can have an extraction blade mounted to a blade stem inside the elongated housing. The blade stem can be attached to a shaft extending through the elongated housing, and the shaft can be coupled to the actuation handle such that an engagement of the actuation handle causes the extraction blade to extend out the extraction opening. Additionally, an internal obstruction (e.g., an opening lip disposed around and inset from the extraction opening) can be operable to push the extraction blade into an angled orientation relative to a centerline of the biopsy sample extraction device when the extraction blade is extended out the extraction opening.

In some examples, the extraction blade is contained in a chamber (e.g., a blade chamber) of the elongated housing when the biopsy sample extraction device is in a resting state (e.g., not being used). The biopsy sample extraction device can further include an opening seal operable to cover the extraction opening and contain a biopsy sample in the blade chamber after performing a biopsy extraction procedure to create the biopsy sample with the extraction blade. Additionally or alternatively, the engagement of the actuation handle includes pushing the actuation handle in a direction towards the extraction opening. The biopsy sample extraction device can further include a first linear channel or spiraling channel or protrusion formed into an interior surface of the elongated housing to mate with a second linear channel or spiraling channel or protrusion on the actuation handle. Furthermore, the first linear or spiraling channel or protrusion can cause the extraction blade to rotate in response to the engagement of the actuation handle. In some instances, a first linear channel or protrusion is formed into an interior surface of the elongated housing to mate with a second linear channel or protrusion on the actuation handle, causing the extraction blade to move in a linear cutting motion.

In some scenarios, the extraction blade is movably mounted to the blade stem via a hinge connecting the extraction blade to the blade stem. A first distance from a centerline of the biopsy sample extraction device to the extraction blade can be greater than a second distance from the centerline to the internal obstruction, such that the extraction blade can abut the opening lip when extended out the extraction opening. Moreover, the blade stem can be attached to the shaft at an attachment point offset from the centerline. A stamp can be disposed on the actuation handle for marking a target biopsy area. The biopsy sample extraction device can further include a vacuum system, fluidly coupled to the extraction opening, to provide suction at the extraction opening and pull a biopsy sample into a sample storage chamber. Additionally, in some instances, the extraction blade is a first extraction blade; the blade stem is a first blade stem; and/or the biopsy sample extraction device further includes a second extraction blade mounted to a second blade stem attached to the shaft.

In some examples, a biopsy extraction device includes a housing with an extraction opening at a distal end. The biopsy extraction device can also include an extraction blade mounted to a blade stem inside the housing. The blade stem can be attached to a shaft extending through the housing and coupling to an actuation handle, such that an engagement of the actuation handle causes the extraction blade to extend linearly out the extraction opening and/or rotate about a centerline of the biopsy extraction device. Additionally, an opening lip disposed around the extraction opening can be operable to push the extraction blade into an angled orientation relative to the centerline of the biopsy extraction device when the extraction blade is extended out the extraction opening.

In some instances, the distal end is a first distal end of the housing; and/or the actuation handle is disposed at a second distal end of the housing opposite the first distal end. The extraction blade can be moveably mounted to the blade stem via a living hinge; and/or the opening lip can be operable to push the extraction blade into the angled orientation by bending the living hinge. Furthermore, the biopsy extraction device can include a chamber at the distal end of the housing fluidly coupled to the extraction opening, the chamber containing: the extraction blade when the biopsy extraction device is in a resting state; and/or a biopsy sample upon performing a biopsy extraction procedure with the biopsy extraction device.

In some examples, a method of extracting a biopsy sample includes placing an extraction opening, at a first distal end of a biopsy extraction device, over a target biopsy area; and/or engaging an actuation handle at a second distal end of the biopsy extraction device, the actuation handle attached to a shaft such that engaging the actuation handle causes an extraction blade coupled to the shaft via a blade stem to extend out the extraction opening. The method can further include changing an orientation of the extraction blade relative to a centerline of the biopsy extraction device using an opening lip disposed around the extraction opening; and/or forming the biopsy sample at the target biopsy area with the extraction blade via a linear motion and/or a rotation of the extraction blade caused by the shaft. In some scenarios, the method includes marking the target biopsy area with a stamp disposed on the biopsy extraction device. Furthermore, forming the biopsy sample can include giving the biopsy sample an asymmetrical profile as an orientation indicator. Additionally or alternatively, forming the biopsy sample can use one or more linear cuts and/or rotational cuts with the extraction blade(s) and can omit an additional cut for separating the biopsy sample from the target biopsy area.

Furthermore, a biopsy sample extraction device can include a compression-based actuation system. The compression-based actuation system can include a rotatable blade disposed at an interior of a housing and coupled to an end of a spring; a trigger bar configured to contact the rotatable blade under tension provided by the spring; and/or a sliding end having one or more actuation members which move the trigger bar responsive to moving the sliding end. The one or more locking mechanisms can include at least one of: a protective cap with a locking post configured to prevent motion of the trigger bar when the protective cap is at least partially disposed over an extraction opening; or a locking clip configured to prevent a sliding motion of the sliding end.

In some examples, the one or more actuation members can extend from an inner surface of the sliding end and can include one or more detents corresponding to one or more living hinges on a mating end of a housing. The one or more detents can include a first detent spaced a distance apart from a second detent, the first detent is configured to mate with a protrusion of the one or more living hinges to at least partially immobilize the sliding end when the biopsy sample extraction device is in a ready position, and the second detent is configured to mate with the protrusion to at least partially immobilize the sliding end after actuating the rotatable blade. Furthermore, the rotatable blade can include a channel for receiving an actuation connector, the channel can be formed at a side of the rotatable blade bent inward. The rotatable blade can be formed of tubular metal with a spool disposed between sidewalls of the tubular metal. Additionally, the trigger bar can contact the rotatable blade at an edge of the rotatable blade, which is an opposite end from a cutting edge of the rotatable blade, to prevent rotation of the rotatable blade when the biopsy sample extraction device is in a ready position. Moreover, the housing is an elongated housing with an elliptical profile. The biopsy sample extraction device can also be transitionable to an armed position by removing at least one of the protective cap or the locking clip.

In some instances, a biopsy sample extraction device includes an elongated housing and/or a sliding end coupled to a mating end of the elongated housing. The biopsy sample extraction device can also include a compression-based actuation system including a rotatable blade coupled to a spring disposed in the elongated housing, the rotatable blade at least partially held in place by a trigger bar, and configured such that a force against a front face of the sliding end causes the trigger bar to release the rotatable blade; and/or a dual-action locking system including one or more removable obstructions to prevent the trigger bar and the sliding end from moving.

In some instances, the one or more removable obstructions include at least a locking clip and a protective cap. The protective cap can include a locking post, extending from an inner surface of the protective cap, configured to occupy a space adjacent the trigger bar when the protective cap is disposed on the biopsy sample extraction device. Moreover, the locking post can include a slanted end that faces inward with respect to a centerline of the elongated housing. The slanted end can be a first slanted end; and/or the sliding end can include one or more actuation members with one or more second slanted ends. The one or more second slanted ends can face outward with respect to the centerline of the elongated housing. In some instances, the second slanted ends face in an opposite direction or a different direction than the first slanted ends.

In some examples, the one or more removable obstructions include a locking clip having a first arm and a second arm. The first arm and the second arm can be curved to correspond to a shape of the elongated housing. Additionally, the locking clip can include a pull tab coupled the first arm and the second arm. The locking clip can also be disposed adjacent to a lip which is positioned at least partially circumferentially around the elongated housing when the locking clip is preventing motion of the sliding end. Furthermore, one or more guide channels can be defined into a mating end of the elongated housing for slidably mating with the sliding end.

In some scenarios, a method of forming a biopsy sample extraction device includes securing a rotatable blade in a locked position in a housing of the biopsy sample extraction device, the rotatable blade being coupled to a compressed spring disposed in the housing; forming a slidable coupling between a sliding end and the housing; positioning, as a first locking mechanism, a protective cap over at least a portion of the sliding end; and/or positioning, as a second locking mechanism, a locking clip between the sliding end and the housing. The method can further include configuring the biopsy sample extraction device to create a biopsy sample with a rotational cut of the rotatable blade by: configuring the protective cap to be pulled away by using a first force in a first direction; configuring the locking clip to be pulled away by using a second force in a second direction; and/or configuring the sliding end to receive a force which causes a living hinge to release a detent on the sliding end, thus releasing the compressed spring and causing the rotatable blade to rotate about a pin.

The foregoing is intended to be illustrative and is not meant in a limiting sense. Many features of the embodiments may be employed with or without reference to other features of any of the embodiments. Additional aspects, advantages, and/or utilities of the presently disclosed technology will be set forth in part in the description that follows and, in part, will be apparent from the description, or may be learned by practice of the presently disclosed technology.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate implementations of systems, methods, and devices consistent with the disclosed subject matter and, together with the description, serves to explain advantages and principles consistent with the disclosed subject matter, in which:

FIG. 1 illustrates an example system for performing a biopsy tissue sample extraction procedure with a biopsy sample extraction device;

FIG. 2 illustrates an example system for performing a biopsy tissue sample extraction procedure with a biopsy sample extraction device including an extraction blade mounted to a blade stem, which can form at least a portion of the system depicted in FIG. 1;

FIG. 3 illustrates an example system for performing a biopsy tissue sample extraction procedure with a biopsy sample extraction device including an elongated housing, which can form at least a portion of the system depicted in FIG. 1;

FIG. 4 illustrates an example method of performing a biopsy tissue sample extraction procedure with a biopsy sample extraction device, which can be performed by the system depicted in FIG. 1;

FIG. 5A illustrates a sectional isometric view of an example biopsy tissue sample extraction device with a compression-based actuation system, which can form at least a portion of the system depicted in FIG. 1;

FIG. 5B illustrates a sectional side view of an example biopsy tissue sample extraction device with a compression-based actuation system, which can form at least a portion the system depicted in FIG. 5A;

FIG. 5C illustrates an example biopsy tissue sample extraction device with a compression-based actuation system and a dual-action safety configuration, which can form at least a portion of the system depicted in FIG. 5A;

FIG. 5D illustrates example actuation and/or safety components of a biopsy tissue sample extraction device, which can form at least a portion of the system depicted in FIG. 5A;

FIG. 5E illustrates an example rotatable blade of a biopsy tissue sample extraction device, which can form at least a portion of the system depicted in FIG. 5A;

FIG. 5F illustrates an example biopsy tissue sample extraction device with a button-based actuation system, which can form at least a portion of the system depicted in FIG. 5A; and

FIG. 6 illustrates an example method of performing a biopsy tissue sample extraction procedure with a biopsy sample extraction device using a compression-based actuation system, which can be performed by the system(s) depicted in FIGS. 1 and/or 5A.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” “forward,” “backward,” and “side,” are used in the description for clarity in specific reference to the figures and are not intended to limit the scope of the presently disclosed technology or the appended claims. Further, it should be understood that any one of the features of the presently disclosed technology may be used separately or in combination with other features.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the presently disclosed technology may include a variety of combinations and/or integrations of the embodiments described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

The systems, methods, and devices disclosed herein include a biopsy sample extraction device to form and extract a tissue biopsy sample quickly, consistently, and safely. The biopsy sample extraction device can have an elongated housing with an extraction opening at a first distal end and an actuation handle at a second distal end. An extraction blade mounted to a blade stem can attach to a shaft running through the biopsy sample extraction device, which couples the blade stem to the actuation handle. This interconnected arrangement of components inside the biopsy sample device causes motion of the actuation handle (e.g., at the first distal end) to translate into motion of the extraction blade (e.g., at the second distal end). Pushing/rotating the shaft with the actuation handle can cause the extraction blade to extend out the extraction opening in a linear cutting motion and/or to rotate in a circular, cutting motion.

Furthermore, when the extraction blade extends out the extraction opening, an internal obstruction (e.g., an opening lip disposed around the extraction opening) can press against the extraction blade, or the blade stem to which the extraction blade is mounted, pushing the extraction blade toward a center line of the biopsy sample extraction device and changing an approach angle (e.g., an orientation angle) of the extraction blade. The approach angle of the extraction blade can cause the biopsy sample to be cut in a single cutting motion. In some instances, multiple blades can move in a linear motion with an approach angle that causes the multiple blades to meet at a point. Additionally or alternatively the blade(s) can include a rotational motion. As such, the biopsy sample can be cut into a shape that is conical, hexagonal, pyramidal, square pyramidal, tetrahedral, or any shape with a curved or multiple flat surfaces terminating at a point. In some instance, a conical shaped sample is formed in examples that use a shallower cut, (e.g., performed by non-medical personnel and/or self-induced) whereas the pyramidal shaped sample is formed with deeper cuts (e.g., performed by medical personnel). Linear cut motions can form the pyramidal shaped samples, and/or rotational cut motions can form the conical shaped biopsy. The extraction blade can be pulled back into the biopsy sample extraction device, which pulls the severed biopsy sample into a chamber or vial contained in the biopsy sample device. The biopsy sample device disclosed herein can include additional features, such as a vacuum system to assist in pulling the biopsy sample into the biopsy sample device/vial for storage and transportation, and/or a stamp to mark a target tissue area with non-toxic ink for improved alignment with the extraction opening.

These biopsy extraction and collection techniques can replace the multi-step process performed by multiple medical professionals with a highly accurate, single-step process. The biopsy sample extraction device can create and extract the biopsy sample quickly, consistently, and safely. The motion of the extraction blade makes a single cut that fully severs the biopsy sample tissue from the surrounding tissue of the target area. The single engagement action to punch, severe, retrieve, and store the biopsy sample is simple such that the biopsy sample extraction device, in some scenarios, is operable by a single operator without an assistant. The operator can be the patient using the biopsy sample device on him/herself and/or someone without medical training. Safety is improved because the extraction blade extending and retracting from the extraction opening replaces the use of any additional cuts at the base of the biopsy sample tissue with a scalpel/forceps. The extraction blade is safely stored in an internal chamber when the actuation handle is not engaged. Furthermore, the biopsy extraction device itself can store the biopsy sample (e.g., using an opening seal to cover the extraction opening), such that the patient may be able to mail the biopsy sample to a laboratory for analysis. Accordingly, the biopsy extraction procedure can be performed remotely, away from a clinic at a home of the patient, and/or at an isolated/remote location with minimal available health services.

Moreover, the quality of the biopsy sample is improved because handling of the biopsy sample is minimized, which preserves the physical characteristics of the biopsy sample. The conical or asymmetrical profile of the biopsy sample can provide an orientation indicator for the laboratory analysis performed on the biopsy sample, such that the laboratory is able to associate a side or direction with the biopsy sample (e.g., top, bottom, right side, left side, etc.). In some instances, the biopsy sample device can create a standardized biopsy sample shape and/or size, which can be aggregated with a large amount of similarly created biopsy samples in laboratory analysis databases for further data analysis using large data sets. Accordingly, the laboratory analysis results are improved using the techniques discussed herein.

Additionally, the consistent, simplified procedure can result in a more precise and accurate incision. The biopsy tissue sample can be formed with a smaller or eliminated margin/buffer zone—significantly reducing the size of the biopsy sample—which improves patient healing and reduces scars and blemishes.

Additional advantages of the technology disclosed herein will become apparent from the detailed description below.

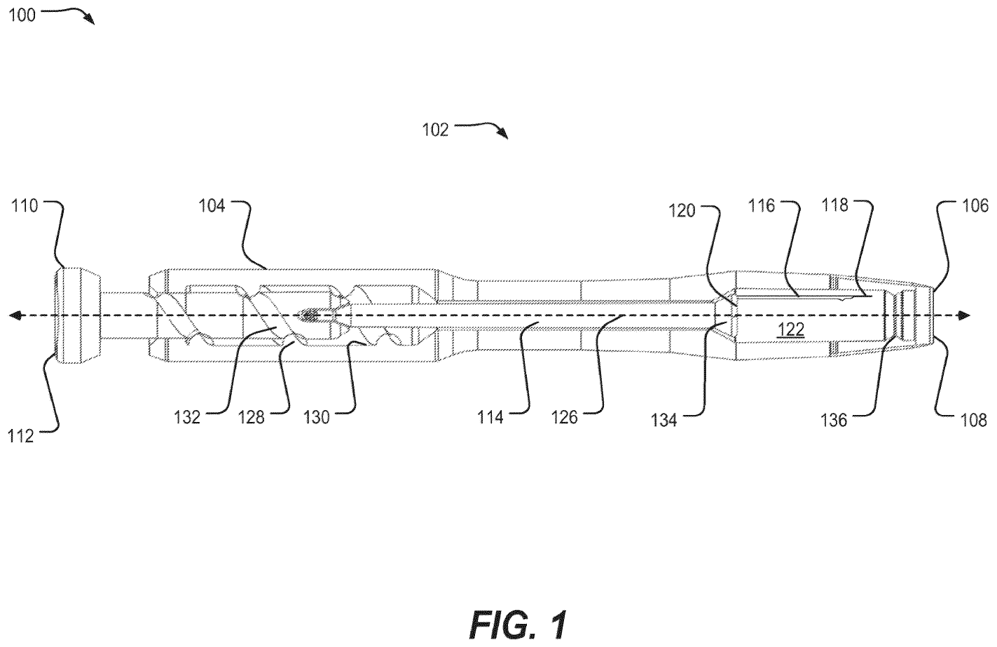

FIG. 1 illustrates an example system 100 for performing a biopsy tissue sample extraction procedure with a biopsy sample extraction device 102. The biopsy sample extraction device 102 can include an elongated housing 104 with an extraction opening 106 formed at a first distal end 108. An actuation handle 110 can be formed at a second distal end 112 (e.g., opposite the first distal end 108). The actuation handle 110 can couple to a shaft 114 which, in turn, couples to a blade stem 116 to which an extraction blade 118 is mounted. The shaft 114 can have a first shaft end 120 terminating in a chamber 122 proximate to the first distal end 108. Additionally, the shaft 114 can have a second shaft end 124 coupled to the actuation handle 110 (e.g., at a sliding and/or rotating portion of the actuation handle 110).

In some examples, a biopsy tissue sample extraction procedure can include an engagement or actuation of the actuation handle 110. This can cause the actuation handle 110 to move along an axis of a centerline 126 (e.g., a length dimension) of the biopsy sample extraction device 102. The engagement of the actuation handle 110 can also cause a linear motion and/or a rotation of the shaft 114 due to frictional forces of a protrusion 128 extending from an interior surface 130 of the elongated housing 104 mating with a channel 132 formed into a body of the actuation handle 110. It is to be understood that, additionally or alternatively, the protrusion 128 can be formed into the body of the actuation handle 110 to mate with the corresponding channel 132 formed into the interior surface 130 of the elongated housing 104. The engagement or actuation along the length dimension can, accordingly, cause the shaft 114 to move along the length dimension and/or can subsequently or simultaneously rotating about the axis of the centerline 126. In some instances, a combination of various channels 132 and/or protrusions 128 can facilitate additional motions for a multi-stage engagement. For instance, a first motion of the shaft 114 in the length dimension can be followed by a second, rotational motion of the shaft 114. In some instances, one or a plurality of blades move only in a linear motion to form the biopsy tissue sample 202

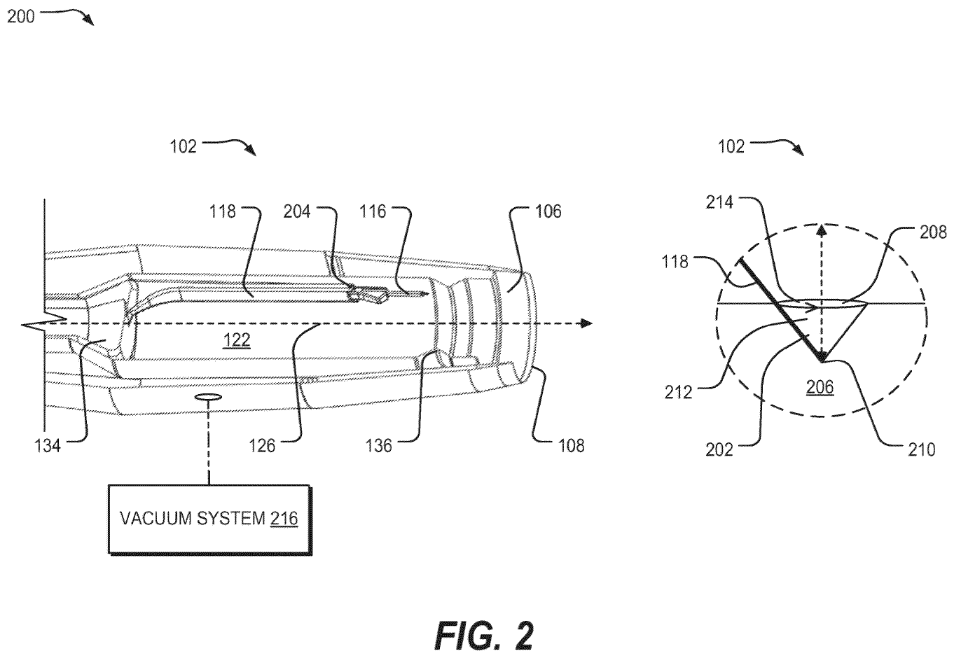

In some instances, the engagement or actuation of the actuation handle 110, and the corresponding motion of the shaft 114, cause the extraction blade 118 to move in a biopsy extracting motion (as discussed in greater detail below regarding FIG. 2). The blade stem 116 to which the extraction blade 118 is mounted can attach to the shaft 114 at the first shaft end 120 offset from the centerline 126. For instance, the first shaft end 120 can include a flared head 134, and the blade stem 116 can couple to the shaft 114 at an outer edge of the flared head 134. In this configuration, a rotational motion of the shaft 114 can cause the first shaft end 120 to spin such that the blade stem 116, and the mounted extraction blade 118 move in a circular motion. Additionally or alternatively, the multiple extraction blades 118 can move in a linear motion caused by a linear motion of the shaft 114.

Furthermore, the extraction blade 118 can be contained in the chamber 122 when the biopsy sample extraction device 102 is in a resting state (e.g., with the actuation handle 110 being engaged or actuated). In other words, the chamber 122 can be an extraction blade storage chamber. Moreover, upon forming and collecting a biopsy tissue sample, the chamber 122 can store the biopsy tissue sample. As such, the chamber 122 can also be a biopsy sample storage container. In this regard, the chamber 122 can be a multi-functional chamber for storing both the extraction blade 118 (e.g., to prevent finger punctures and improve safety) and the collected biopsy tissue sample (e.g., to prevent contamination of the biopsy tissue sample and/or maintain an orientation and structural integrity of the biopsy tissue sample). Additionally, the biopsy sample extraction device 102 can include an internal obstruction such as an opening lip 136 formed around the extraction opening 106. The opening lip 136 can include a protrusion and/or ring formed into the interior surface 130 an inset distance from the outer edge of the extraction opening 106. The opening lip 136 can be formed inside the biopsy sample extraction device 102 and can form a reduced/tapered interior diameter where the chamber 122 transitions to the extraction opening 106. As such, opening lip 136 can abut against the extraction blade 118 and/or the blade stem 116 and change an angle orientation of the extraction blade 118 when the extraction blade 118 is extended out the extraction opening 106 (e.g., in response to the engagement of the actuation handle 110). This biopsy extraction and collection process is discussed in greater detail below.

FIG. 2 illustrates an example system 200 for performing the biopsy tissue sample extraction procedure with the biopsy sample extraction device 102. The biopsy sample extraction device 102 can include the extraction blade 118 coupled to the blade stem 116 to form the biopsy tissue sample 202. The system 200 depicted in FIG. 2 can form at least a portion of the system 100.

In some instances, the extraction blade 118 is pushed and/or extends out of the extraction opening 106 in response to the engagement of the actuation handle 110. A first motion along the length dimension of the centerline 126 can cause the extraction blade 118 (e.g., a back side of the extraction blade 118) and/or the blade stem 116 (e.g., a back side of the blade stem 116) to contact or abut against the opening lip 136. In some instances, the extraction blade 118 and/or the blade stem 116 can slide along a track formed into an inner surface of the chamber 122. Additionally or alternatively, the internal obstruction forming the orientation angle can include a sloped surface of the elongated housing 104. Accordingly, as the extraction blade 118 is slid forward and out the extraction opening 106, the internal obstruction (e.g., the opening lip 136) can exert a force on the extraction blade 118 and/or the blade stem 116 in a direction towards the centerline 126, causing the extraction blade 118 to be oriented at an angle towards the centerline 126. The angle of the extraction blade 118 with respect to the centerline 126 can be changed from parallel with the centerline 126 to non-parallel with the centerline 126 (e.g., pointed towards the centerline 126). This change in the orientation angle can be created by the opening lip 136 pushing against the extraction blade 118 and/or the blade stem 116 and causing the extraction blade 118 to pivot at a hinge 204. The hinge 204 can connect the extraction blade 118 to the blade stem 116. Additionally or alternatively, the hinge 204 can be a living hinge formed into the blade stem 116. For instance, the blade stem 116 can be formed of a flexible material that is bent by the force exerted by the opening lip 136. Additionally or alternatively the extraction blade 118 can have the angle towards the centerline 126 formed simply by the angle at which the extraction blade 118 attaches to the blade stem 116, or by other mechanisms, such as a rod attaching a portion of the blade stem 116 to another component of the biopsy sample extraction device 102 (e.g., a stationary portion such as the interior surface 130) such that the rod pulls the blade stem 116 toward the centerline 126 when the shaft 114 moves forward.

The orientation angle of the extraction blade 118 being non-parallel to the centerline 126 can result in creating a biopsy tissue sample 202 that is fully severed and/or fully removable from surrounding tissue 206. For instance, the extraction blade 118 can be inserted into a target tissue area 208 forming an acute angle with the centerline 126. A tip of the extraction blade 118 can terminate at a point 210 inside the patient that defines a distal tip of the biopsy tissue sample 202. The extraction blade 118 can make a linear cut corresponding to the linear motion of the shaft 114. Moreover, the extraction blade 118 can make a rotational cut (e.g., in a circular motion) corresponding to the rotational motion of the shaft 114. The linear cut and/or rotational cut at the acute angle can fully excise the biopsy tissue sample 202 from the target tissue area 208, for instance, by maintaining the tip of the 118 at the point 210 inside the patient as the extraction blade 118 is rotated and cuts the biopsy tissue sample 202, or by meeting multiple linearly moving blades at the point 210. Once the cutting motion(s) are complete (e.g., caused by the single actuation of the actuation handle 110), the biopsy tissue sample 202 can be fully severed from the target tissue area 208, and can be extracted from the surrounding tissue 206, for instance, without requiring any additional cuts. In other words, only the forward, linear, and/or rotational movements of the extraction blade 118 (e.g., corresponding to the engagement of the 110) are used to form the biopsy tissue sample 202, omitting reliance on any additional cuts for severing the biopsy tissue sample 202 from the surrounding tissue 206 using scalpels or forceps.

The biopsy tissue sample extraction techniques performed with the biopsy sample extraction device 102 can form the biopsy tissue sample 202 with an asymmetrical profile 212. For instance, a top portion of the biopsy tissue sample 202 can have a flat, circular surface 214, and a bottom portion of the biopsy tissue sample 202 can be formed to terminate at the point 210, such that the biopsy tissue sample 202 has a conical shape. Accordingly, the top portion can be asymmetrical relative to the bottom portion. This asymmetrical profile 212 can be an orientation indicator for the biopsy tissue sample 202 when the biopsy tissue sample 202 is sent to a lab for a biopsy tissue analysis procedure. For instance, the flat, circular portion can indicate a top (e.g., exposed, outer surface) of the biopsy tissue sample 202, and the termination point 210 can indicate a bottom or deepest portion of the biopsy tissue sample 202. In other words, the biopsy tissue sample 202 can have a conical shape which is asymmetrical about a horizontal axis. The dimensions of the biopsy tissue sample 202 can be defined by the movement distance of the actuation handle 110 and the shaft 114, as well as the orientation angle of the extraction blade 118. In some instances, the biopsy tissue sample 202 can have a depth of between 5 mm and 25 mm. Furthermore, in some instances, the depth can be between 3 mm and 33 mm. Furthermore, the biopsy sample extraction device 102 can include a depth lock and/or a width lock for setting a predefined depth and/or predefined width of the extraction blade 118 (e.g., via one or more stops, protruding from the interior surface 130, to engage the actuation handle 110, the shaft 114, the blade stem 116, or other internal components of the biopsy sample extraction device 102). In this way, the biopsy tissue sample 202 can have a precisely-defined, consistent shape without needing the additional 5 mm buffer of tissue to account for human error that previous techniques relied upon. Therefore, the biopsy sample extraction device 102 can create biopsy tissue sample 202 that results in less scar tissue and/or a smaller extraction area (e.g., a smaller tissue “pothole”) than previous techniques.

This orientation indicator function can also be useful for various tissue analyses performed at the lab. For instance, an analysis of tissue health can use the asymmetrical profile to determine or recreate the orientation of the biopsy tissue sample 202. An analysis of suspicious tissue (e.g., necrotic tissue, infected tissue, cancerous tissue, diseased tissue, malignant tissue, benign tissue, a wart, unwanted tissue, discolored tissue, sus′ tissue, and/or the like) can determine a tissue feature (e.g., an infected portion, a cancerous portion, a necrosis portion, etc.) and/or a gradient of the tissue feature directionally from one side of the biopsy tissue sample 202 to the other, and/or from a first depth to a second depth, and the like. In this way, a maximum amount of information that can be gleaned from the biopsy tissue sample 202 is maintained from the initial extraction process throughout the lifetime of the biopsy tissue sample 202 as it is transported to the lab analysis due to the predefined shape of the biopsy tissue sample 202. The orientation of the biopsy tissue sample 202 can be recreated in the lab setting to determine which side is an outer surface side, which side is an inner tissue side, which side is proximate to other organs of the patient, a left side, a right side, and the like. Moreover, storage of the biopsy tissue sample 202 (e.g., in the chamber 122 and/or a vial, as discussed below) further preserves the characteristics and useful information of the biopsy tissue sample 202 by reducing the number of handling touch points between biopsy tissue sample formation/extraction and lab analysis.

In some instances, the biopsy sample extraction device 102 can include a plurality of extraction blades 118 for cutting the biopsy tissue sample 202. For instance the extraction blade 118 can be a first extraction blade, and the biopsy sample extraction device 102 can further include a second extraction blade, a third extraction blade, a fourth extraction blade, etc. In embodiments with a plurality of extraction blades 118, the extraction blades 118 can be evenly or uniformly spaced apart about the outer edge of the flared head 134 at the first shaft end 120. For instance, the biopsy sample extraction device 102 can include two, three, four five, etc. blade stems 116 extending from the 120 in a circular arrangement, with the plurality of blade stems mounting the plurality of extraction blade 118 (e.g., via a one-to-one correspondence). The plurality of extraction blade 118 can meet at the point 210 to fully form and severe the biopsy tissue sample 202 from the target tissue area 208. As such, a single engagement of the actuation handle 110 can cause the plurality of extraction blade 118 to move forward, extend out of the extraction opening 106, and move in a linear motion and/or rotate in circular motion to cut the biopsy tissue sample 202 from the surrounding tissue 206. The extraction blade(s) 118 (e.g., and/or the shaft 114 or the actuation handle 110) can be spring-loaded such that a release of the actuation handle 110 causes the extraction blade(s) 118 to retract back into the chamber 122. The extraction blade(s) 118 can be stainless steel surgical blades.

In some examples, the biopsy sample extraction device 102 can include a vacuum system 216 for providing a vacuum pressure at the extraction opening 106, which creates suction at the extraction opening 106. The vacuum system 216 can include a pump and/or one or more valves fluidly coupled to the extraction opening 106 (e.g., via the chamber 122). Additionally or alternatively the vacuum system 216 can include a CO₂ cannister for generating a suction pressure at the extraction opening 106. The vacuum system 216 can be triggered or activated by the engagement of the actuation handle 110 (e.g., during and/or after the biopsy tissue sample 202 is cut from the target tissue area 208), to pull the newly formed biopsy tissue sample 202 into the biopsy sample extraction device 102.

For instance, upon forming the biopsy tissue sample 202 with the extraction blade 118, the biopsy tissue sample 202 can be pulled into the chamber 122, which can act as a biopsy sample storage chamber. The biopsy tissue sample 202 can be pulled into the chamber 122 manually (e.g., by retracting the extraction blade(s) 118) and/or with the assistance of the vacuum system 216). Additionally or alternatively, the biopsy sample extraction device 102 can include a vial (e.g., disposed in the elongated housing 104 or outside and attached to the elongated housing 104) into which the biopsy tissue sample 202 is placed. Furthermore the biopsy sample extraction device 102 can include an opening seal that is placed over the extraction opening 106 after the biopsy tissue sample 202 is formed and collected into the chamber 122, thus converting the chamber 122 into a sample storage chamber (e.g., for biopsy tissue sample 202 storage and/or transport).

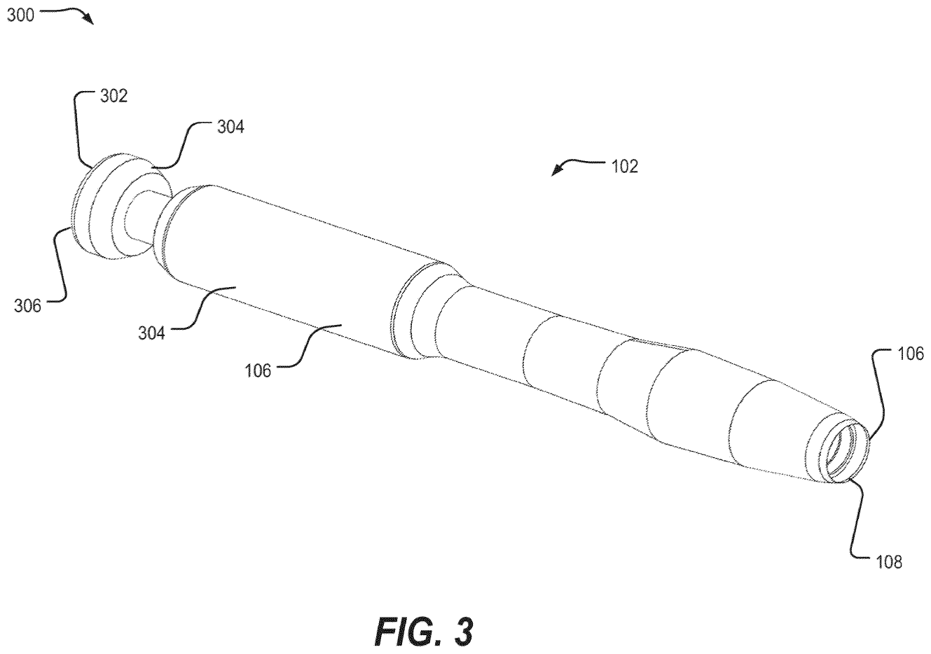

FIG. 3 illustrates an example system 300 for performing the biopsy tissue sample extraction procedure with the biopsy sample extraction device 102. The biopsy sample extraction device 102 can include the elongated housing 104 with the extraction opening 106 at the first distal end 108. The system 300 depicted in FIG. 3 can form at least a portion of the system 100.

The elongated housing 104 can be formed of a rigid material, such as plastic, metal, and the like. For instance, the biopsy sample extraction device 102 can be a disposable device formed of plastic with a metal (e.g., surgical steel) extraction blade 118. In some instances, the elongated housing 104 has a length dimension of between 4 inches and 12 inches. Moreover, the extraction opening 106 can have a width dimension or diameter of between 3 mm and 50 mm. In some scenarios, the biopsy sample extraction device 102 can be operable for insertion into a mouth of a patient to extract an oral biopsy tissue sample 202.

In some examples, the biopsy sample extraction device 102 includes a stamp 302 formed onto an exterior surface 304 of the biopsy sample extraction device 102. For instance, the stamp 302 can be formed on an end 306 of the actuation handle 110 (e.g., at the second distal end 112). The stamp 302 can represent the shape of the biopsy tissue sample 202, such as a circle, with dimensions that correspond to the dimensions of the outer diameter of the biopsy tissue sample 202 formed by the extraction blade 118. The stamp 302 can be used with non-toxic ink to mark the target tissue area 208, and the extraction opening 106 can be aligned with the marked target tissue area 208 to create the biopsy tissue sample 202. This process makes the extraction cuts more accurate and reduces the margin of error, such that the 5 mm buffer around the biopsy tissue sample 202 can be eliminated. Furthermore, the biopsy extraction procedure can include taking a photo of the target tissue area 208 after the target tissue area 208 is marked with the stamp to create a record of the target tissue area 208 (e.g., prior to forming the biopsy tissue sample 202). The photo can be associated with the biopsy tissue sample 202 (e.g., a unique identifier associated with the biopsy tissue sample 202) in one or more databases of the laboratory performing analysis on the biopsy tissue sample 202, increasing the information available to the lab technicians and improving the analysis of the biopsy tissue sample 202.

In some instances, the biopsy sample extraction device 102 can be used to create a standardized biopsy tissue sample procedure resulting in a standardized shape and/or size of the biopsy tissue sample 202. A universal database of tissue samples and biopsies can be created using the biopsy sample extraction device 102 and the biopsy tissue extraction techniques discussed herein, due to the consistency created by the biopsy sample extraction device 102. This results in high-quality biopsy tissue sample data on which the analyses are based. In some examples, the universal database can be accessible via a website or application used by the patient and/or medical personnel to view the data created for the biopsy tissue sample 202 (e.g., the photo of the marked target tissue area 208, the results of the lab analyses, and the like).

The biopsy sample extraction device 102 can include a cap or lid placed over the extraction opening 106 for safety purposes when the biopsy sample extraction device 102 is not being used (e.g., for storage and/or transportation). Additionally or alternatively, the biopsy sample extraction device 102 can include a safety lock to prevent accidental engagements of the actuation handle 110.

In some examples, the biopsy sample extraction device 102 can form at least a part of a kit with other biopsy sample extraction devices 102 having different shapes and/or for forming different biopsy tissue sample 202 sizes or shapes. For instances, a kit of biopsy sample extraction devices 102 can include a first biopsy sample extraction device 102 for forming the biopsy tissue sample 202 with a circular top profile, a second biopsy sample extraction device 102 for forming the biopsy tissue sample 202 with a square top profile, a third biopsy sample extraction device 102 for forming the biopsy tissue sample 202 with a triangular top profile, combinations thereof, and/or the like. Moreover, the plurality of biopsy sample extraction device(s) 102 in the kit can have a same top profile (e.g., the circular top profile), but with different size dimensions. For instance, the first biopsy sample extraction device 102 can form the biopsy tissue sample 202 with a small diameter (e.g., 3 mm), the second biopsy sample extraction device 102 can form the biopsy tissue sample 202 with a medium diameter (e.g., 5 mm), the third biopsy sample extraction device 102 can form the biopsy tissue sample 202 with a large diameter (e.g., 7 mm or greater), and/or the like. The kit can include two biopsy extraction devices 202 with one having a larger cutting diameter than the other. It is to be understood that the kit of biopsy sample extraction devices 102 can include a plurality of biopsy sample extraction devices 102 having any combination of shapes and sizes corresponding to different shaped/sized biopsy tissue samples 202

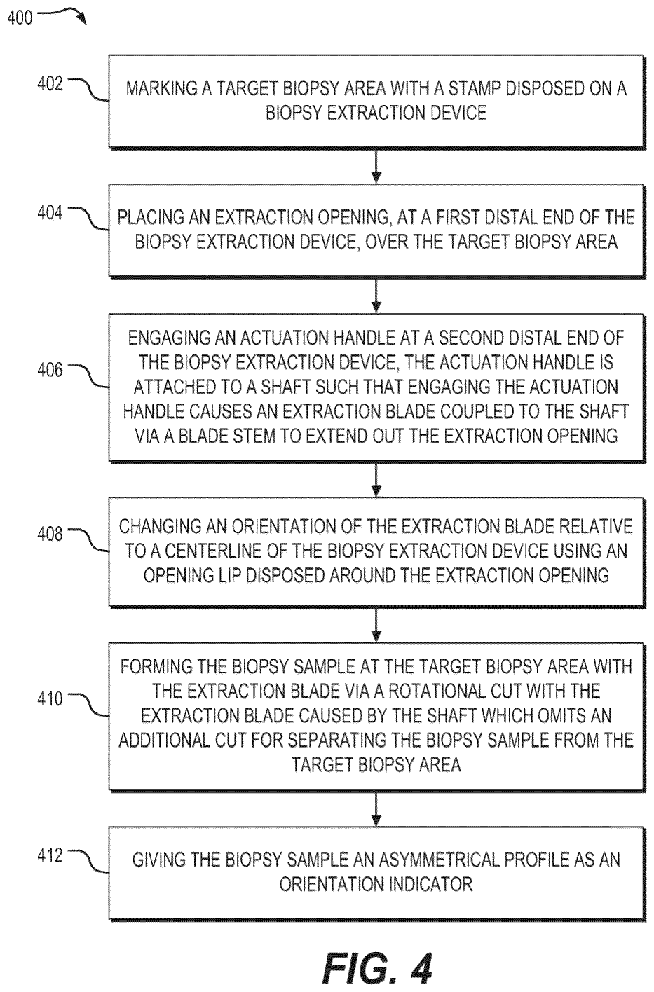

FIG. 4 illustrates an example method 400 for performing the biopsy tissue sample extraction procedure with the biopsy sample extraction device 102. The method 400 can be performed by any of the systems 100-300 discussed herein.

At operation 402, the method 400 can mark a target biopsy area with a stamp disposed on a biopsy extraction device. At operation 404, the method 400 can place an extraction opening, at a first distal end of the biopsy extraction device, over the target biopsy area. At operation 406, the method 400 can engage an actuation handle at a second distal end of the biopsy extraction device, the actuation handle is attached to a shaft such that engaging the actuation handle causes an extraction blade coupled to the shaft via a blade stem to extend out the extraction opening. At operation 408, the method 400 can change an orientation of the extraction blade relative to a centerline of the biopsy extraction device using an opening lip disposed around the extraction opening. At operation 410, the method 400 can form the biopsy sample at the target biopsy area with the extraction blade via a linear cut and/or a rotational cut with the extraction blade caused by the shaft which omits an additional cut for separating the biopsy sample from the target biopsy area. At operation 412, the method 400 can give the biopsy sample an asymmetrical profile as an orientation indicator.

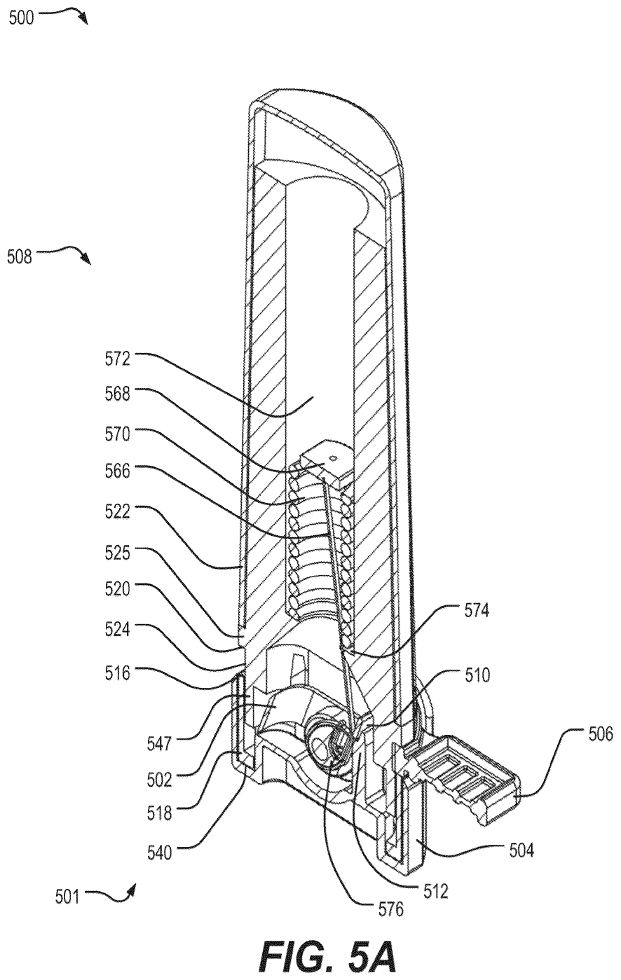

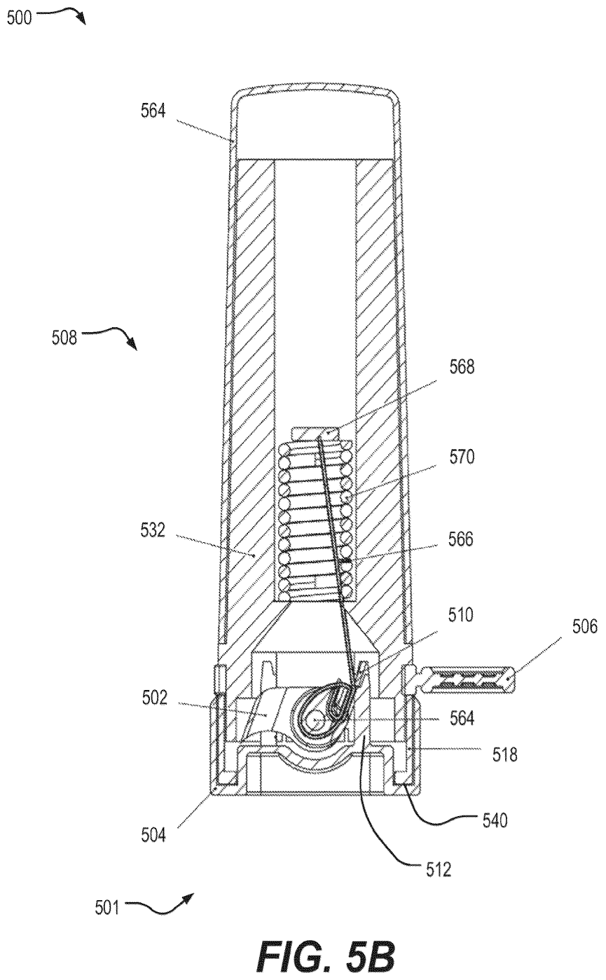

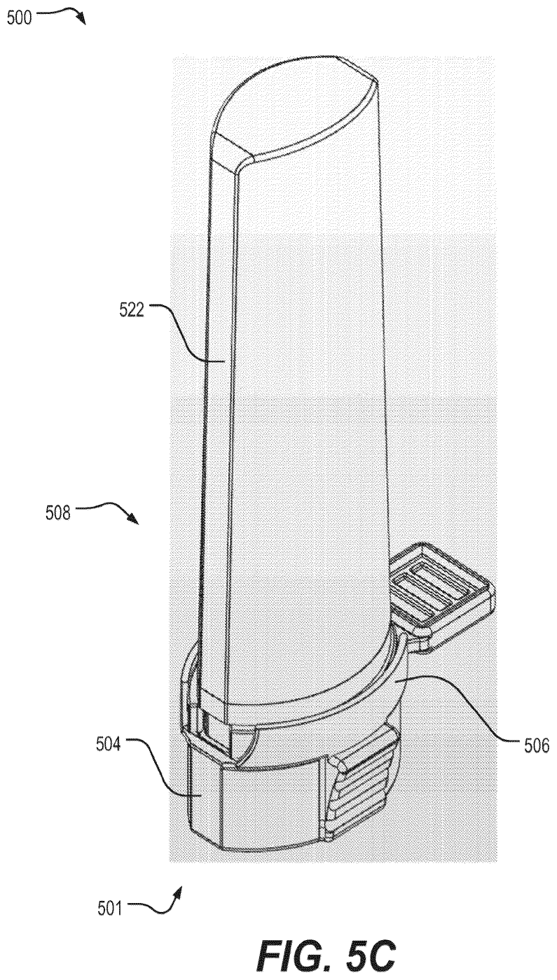

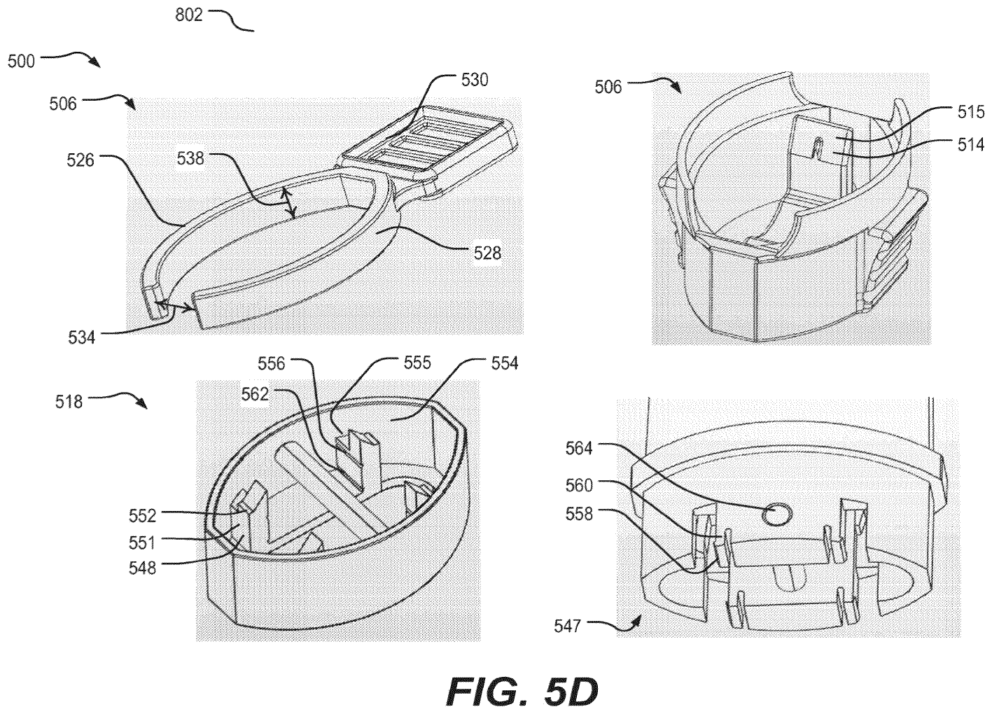

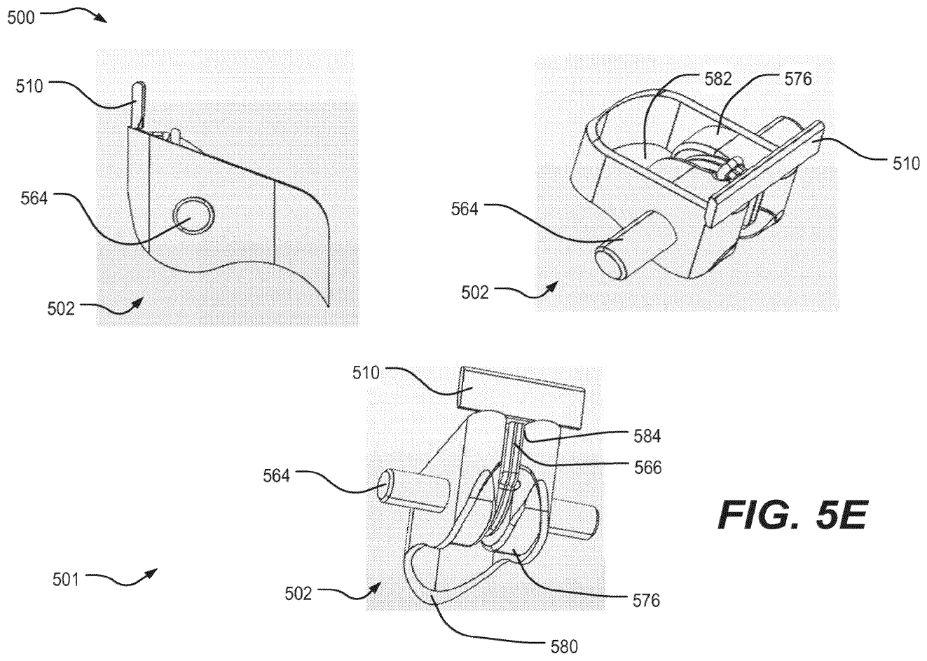

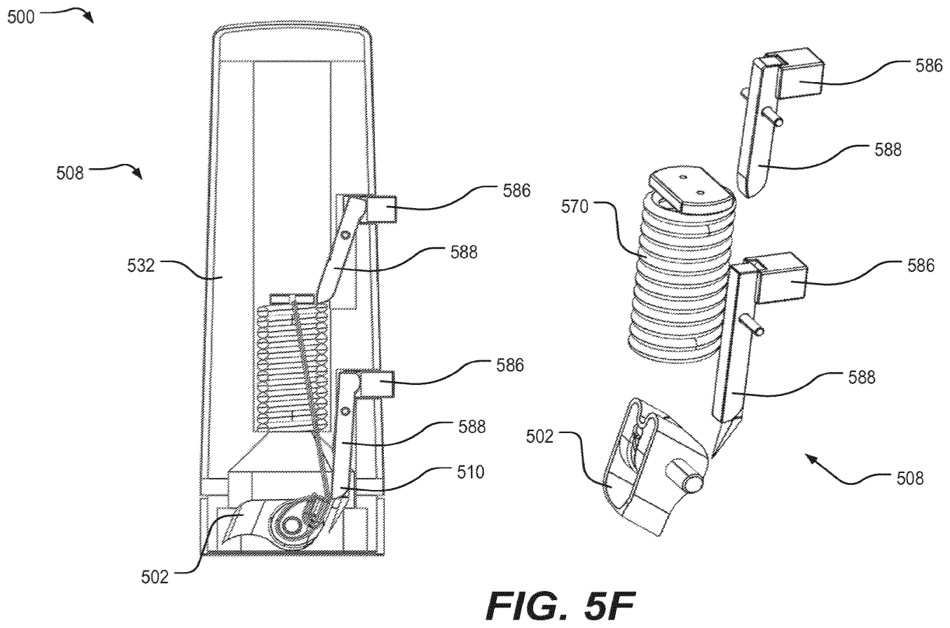

FIGS. 5A-5F illustrates an example system 500 for performing the biopsy tissue sample extraction procedure with the biopsy sample extraction device 102. As depicted in FIGS. 5A-5F, the biopsy sample extraction device 102 can include a rotatable blade 502 and one or more safety or locking features, such as a protective cap 504 and/or a locking clip 506 for securing the rotatable blade 502 in a safe, secured, “ready” position, and transitioning the biopsy sample extraction device 102 to an armed “ready” position (e.g., with compressed or stored kinetic energy ready for actuation). The biopsy sample extraction device 102 depicted in FIGS. 5A-5E can include a compression-based actuation system 501. Additionally or alternatively, as depicted in FIG. 5F, the biopsy sample extraction device 102 can include a button-based actuation system 503. Furthermore, the system(s) 500 depicted in FIGS. 5A-5F can form at least a portion of the system 100.

In some examples, as depicted in FIGS. 5A and 5B, the biopsy sample extraction device 102 includes a dual-action safety configuration 508 in which the biopsy sample extraction device 102 is operable by performing two safety actions—removing the protective cap 504 and removing the locking clip 506. For instance, the rotatable blade 502 can be held in a suppressed or “ready” position (e.g., with the spring compressed) by a trigger bar 510 (e.g., and/or a pin, a protrusion, a flexible stop, a pivoting arm, or the like), which can prevent motion of the rotatable blade 502. The trigger bar 510 can be positioned at one side of the rotatable blade 502 with a bottom edge pressing against a top edge of the rotatable blade 502, such that the trigger bar 510 obstructs a rotational pathway of the rotatable blade 502 when the biopsy sample extraction device 102 is in the “ready” position. To create the dual-action safety configuration 508, the protective cap 504 can include a locking post 512 (e.g., as shown in FIG. 5D) that extends up from a bottom surface of the protective cap 504 to prevent motion of the trigger bar 510, thus locking the rotatable blade 502. The locking post 512 can have a slanted mating surface 514 at its terminating end, which presses against the trigger bar 510 when the protective cap 504 is secured onto the end of the biopsy sample extraction device 102. The slant 515 of the slanted mating surface 514 can be facing in a first direction such that the slanted mating surface 514 faces a center or the centerline 126 of the biopsy sample extraction device 102. The locking post 512 can press against the trigger bar 510 and/or can occupy a space adjacent to the trigger bar 510 such that the trigger bar 510 is locked in place against the rotatable blade 502, unable to move into the space occupied by the protective cap 504. In this position, with the trigger bar 510 unable to move out of the way, the rotatable blade 502 is correspondingly also unable to move. Additionally or alternatively, the locking post 512 can have a cut-out, lip, inset, or seat for the end or corner of the trigger bar 510 to rest against, further securing the trigger bar 510 in place. The locking post 512 can also at least partially occupy or obstruct a space adjacent to the rotatable blade 502 in the rotational pathway of the rotatable blade 502, further preventing movement of the rotatable blade 502. Upon removing the protective cap 504, the locking post 512 can be pulled away from the rotatable blade 502 and the trigger bar 510, clearing the space for the trigger bar 510 to move into, and transitioning the biopsy sample extraction device 102 from the dual-action safety configuration 508 into a single-action safety configuration. In some scenarios, removing (e.g., omitting a locking clip 506) removing the protective cap 504 can transition the biopsy sample extraction device 102 from single-action safety configuration to an armed configuration, or from a “ready and locked” position to a “ready and armed” position. Moreover, removing the protective cap 504 can remove the obstruction of the extraction opening 106 caused by the protective cap 504. Some examples can include multiple trigger bars 510.

The biopsy sample extraction device 102 can also include the locking clip 506, which can provide the second safety feature in the dual-action safety configuration 508 and/or a single-action safety configuration (e.g., with the protective cap 504 removed or omitted). With the locking clip 506 still in place, the compression-based actuation system 501 of the biopsy sample extraction device 102 can be prevented. For instance, the locking clip 506 can be positioned between a first surface 516 of a sliding end 518 of the biopsy sample extraction device 102, and a second surface 520 of an outer housing 522, such as a lip 523, a protrusion, or another portion of the biopsy sample extraction device 102. The sliding end 518 can be prevented from being pushed or slid along an axial direction 525 parallel to the centerline 126 and/or towards the second surface 520. In some scenarios, the locking clip 506 is positioned at least partially circumferentially around a portion of the outer housing 522. For instance, the biopsy sample extraction device 102 can include an indent 524 between the lip 523 wrapping at least partially around the outer housing 522.

In some instances, the locking clip 506 includes a first arm 526 and a second arm 528 extending from a gripping surface 530 (e.g., a gripping or pull tab, a loop, a protrusion, a textured surface, combinations thereof, and the like). The first arm 526 and the second arm 528 can wrap around opposite ends of the outer housing 522, holding the locking clip 506 against the outer housing 522 or other portion of the main body 532. The outer housing 522 can be at least partially circular or elliptical, as shown in FIG. 5C, which can improve gripping comfort. As such, the first arm 526 and the second arm 528 can be curved arms, with the curved portion forming a living hinge. For instance, the shape or curve of the first arm 526 and/or the second arm 528 can correspond to an outer shape, curve, or profile of the main body 532. When the gripping surface 530 is pulled in a direction away from the main body 532, a flex in the first arm 526 and/or the second arm 528 can allow a gap distance 534 between the first arm 526 and the second arm 528 (e.g., between their ends) to widen as they slide over and are pushed apart by the wider central section of the main body 532. In this way, the locking clip 506 can be pulled away with a predefined amount of force (e.g., calibrated based on a material flexibility/rigidity, shape, and/or width of the first arm 526 and/or second arm 528). Accordingly, the locking clip 506 can be secured in place with enough force to avoid accidental removal, while still being removable with a simple finger pull. Furthermore, because removal of the obstructing locking clip 506 can create the clearance in a gap 536 between the first surface 516 and the sliding end 518, a width 538 of the locking clip 506 can correspond to a sliding distance of the sliding end 518. By adjusting the width 538, one can adjust how far the sliding end 518 slides once the locking clip 506 is removed.

In some examples, once the protective cap 504 and the locking clip 506 are separated from the main body 532, the biopsy sample extraction device 102 is in an “armed and ready” position and can be triggered to actuate the rotatable blade 502. The actuation mechanism can be compression-based by contacting the front face 540 of the sliding end 518 against the target tissue area 208 and pushing the biopsy sample extraction device 102 against the skin. The sliding motion of the sliding end 518 can be guided by one or more sliding channels 546 formed into a mating end 547 of the biopsy sample extraction device 102. As depicted in FIG. 5D, the sliding end 518 can have one or more actuation members 548 (e.g., posts, protrusions, springs, or so forth) extending from an inner lip 550 of the sliding end 518. The actuation member(s) 548 can have a wider lower portion 551 with a narrower upper portion 552 separated from an inner wall 554 of the sliding end 518 by a separation gap 555. In some scenarios, the actuation member(s) 548 mate with the sliding channels 546. A first detent 556 in the wider lower portion 551 can mate with a corresponding protrusion 558 on a living hinge 560 defining a portion of the sliding channel 546. Additionally, a second detent 562 can be positioned a separation distance from the first detent 556 to also catch the protrusion 558. In this way, the sliding end 518 can be locked in place by the first detent 556 such that compression against the front face 540 causes the living hinge 560 to flex and the protrusion 558 to slide out of place. A resistive force can be created by this configuration that can be overcome by a predetermined amount of actuation force to cause the actuation. Then the sliding end 518 can slide towards the main body 532 so that the actuation member 548 engages the trigger bar 510. The second detent 562 can catch the protrusion 558 to lock the sliding end 518 in the compressed position after the actuation and/or to prevent the sliding end 518 from moving back in the opposite direction. The actuation member(s) 548 can push the trigger bar 510 to the side so that the trigger bar 510 no longer contacts the rotatable blade 502. The protrusion 558 can have a slant, which can open or face towards the outer wall, (or in a direction opposite the slant 515 of the slanted mating surface 514), to push the trigger bar 510 out of the way of the rotatable blade 502 and create clearance for it to move, thus releasing the tension on the spring. The biopsy sample extraction device 102 can include two actuation members(s) 548, four actuation member(s) 548, or other numbers of actuation member(s) 548.

In some examples, this compression of the sliding end 518 causes the blade actuation by rotating the rotatable blade 502 around a blade pin 564. Prior to the actuation, the rotatable blade 502 can be in a loaded or compressed state. An actuation connector 566 (e.g., a cable, a strip, a pin, or the like) can couple to the rotatable blade 502 at a first end, and to a pull plate 568 at a second end. The pull plate 568 can be disposed on an opposite side of a spring 570 from the 502, holding the spring compressed via the tension of the actuation connector 566. The spring 570 can be sandwiched between the pull plate 568 and a lip or protrusion 574 extending from an inner side wall of the main body 532. In response to the trigger bar 510 being cleared by the actuation member 548, the spring 570 can release and expand into the inner cavity 572 of the main body 532, causing the actuation connector 566 to pull the end of the rotatable blade 502 and rotate it about the blade pin 564

Furthermore, the rotatable blade 502 can have a spool 576, such as a non-circular spool or a tear-shaped spool, for spooling the first end of the actuation connector 566. As shown in FIG. 5D, the rotatable blade 502 can include a rounded edge 580 having a beak or scoop shape (e.g., formed of cut and bent tubular metal). The rotatable blade 502 can include a collection area 582 adjacent to the spool for collecting and/or temporarily storing the biopsy sample. In some examples, the rotatable blade 502 is formed of cut tubular metal with one side, opposite the rounded edge 580, folded in to create a channel 584 for the actuation connector 566. This configuration can create a rotational angle for the rotatable blade 502 greater than 90° and/or greater than 180° to make a complete cut fully severing the biopsy sample.

In some examples, the biopsy sample extraction device 102 can include other variations. For instance, the outer housing 522 can be omitted and/or the inner cylindrical shaft can act as the outer housing. Moreover, as shown in FIG. 5F, the biopsy sample extraction device 102 can include one or more stop button actuators 586 for creating the clearance for the rotatable blade 502 to rotate and/or the spring 570 to expand (e.g., using hinged arm stops 588). Additionally or alternatively, the biopsy sample extraction device 102 can have squeeze-based actuation using the buttons, a flexible outer housing, and/or one or more living hinges. Moreover, other types of blade configurations with different edge shapes may be coupled to the end of the actuation connector 566 for making other shaped or sized biopsy tissue samples (e.g., with one or more interchangeable blades).

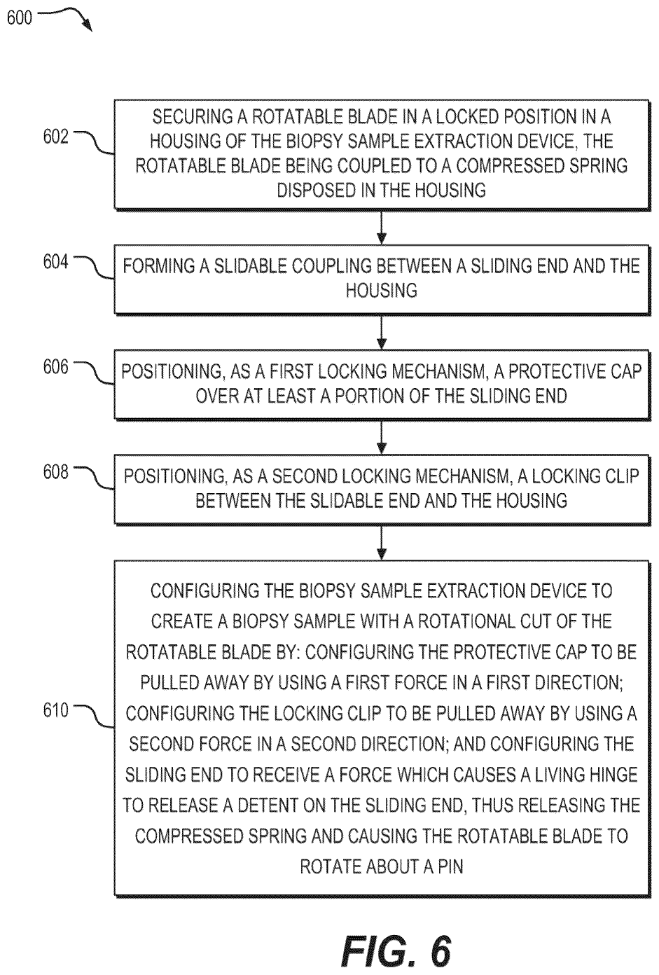

FIG. 6 depicts an example method 600 for collecting a biopsy sample using the biopsy sample extraction device 102, such as the compression-based biopsy sample extraction device 102 depicted in FIGS. 5A-5F.

In some examples, at operation 602, the method 600 can secure a rotatable blade in a locked position in a housing of the biopsy sample extraction device, the rotatable blade being coupled to a compressed spring disposed in the housing. At operation 604, the method 600 can form a slidable coupling between a sliding end and the housing. At operation 606, the method 600 can position, as a first locking mechanism, a protective cap over at least a portion of the sliding end. At operation 608, the method 600 can position, as a second locking mechanism, a locking clip between the sliding end and the housing. At operation 610, the method 600 can configuring the biopsy sample extraction device to create a biopsy sample with a rotational cut of the rotatable blade by: configuring the protective cap to be pulled away by using a first force in a first direction; configuring the locking clip to be pulled away by using a second force in a second direction; and configuring the sliding end to receive a force which causes a living hinge to release a detent on the sliding end, thus releasing the compressed spring and causing the rotatable blade to rotate about a pin.

It is to be understood that the specific order or hierarchy of steps in the method(s) 400 depicted in FIGS. 4 and 6 and throughout this disclosure are instances of example approaches and can be rearranged while remaining within the disclosed subject matter. For instance, any of the operations depicted in FIGS. 4 and 6 and throughout this disclosure may be omitted, repeated, performed in parallel, performed in a different order, and/or combined with any other of the operations depicted in FIGS. 4 and 6 and throughout this disclosure. For instance, operations 410 and 412 can be combined as a single operation or action.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A biopsy sample extraction device comprising:
a compression-based actuation system including:
a rotatable blade disposed at an interior of a housing and coupled to an end of a spring;
a trigger bar configured to contact the rotatable blade under tension provided by the spring; and
a sliding end having one or more actuation members which move the trigger bar responsive to moving the sliding end; and
one or more locking mechanisms including at least one of:
a protective cap with a locking post configured to prevent motion of the trigger bar when the protective cap is at least partially disposed over an extraction opening; or
a locking clip configured to prevent a sliding motion of the sliding end.

2. The biopsy sample extraction device of claim 1,
wherein,
the one or more actuation members extend from an inner surface of the sliding end and include one or more detents corresponding to one or more living hinges on a mating end of a housing.

3. The biopsy sample extraction device of claim 2,
wherein,
the one or more detents include a first detent spaced a distance apart from a second detent, the first detent is configured to mate with a protrusion of the one or more living hinges to at least partially immobilize the sliding end when the biopsy sample extraction device is in a ready position, and the second detent is configured to mate with the protrusion to at least partially immobilize the sliding end after actuating the rotatable blade.

4. The biopsy sample extraction device of claim 1,
wherein,
the rotatable blade includes a channel for receiving an actuation connector, the channel formed at a side of the rotatable blade bent inward.

5. The biopsy sample extraction device of claim 1,
wherein,
the rotatable blade is formed of tubular metal with a spool disposed between sidewalls of the tubular metal.

6. The biopsy sample extraction device of claim 1,
wherein,
the trigger bar contacts the rotatable blade at an edge of the rotatable blade, which is an opposite end from a cutting edge of the rotatable blade, to prevent rotation of the rotatable blade when the biopsy sample extraction device is in a ready position.

7. The biopsy sample extraction device of claim 1,
wherein,
the housing is an elongated housing with an elliptical profile.

8. The biopsy sample extraction device of claim 1,
wherein,
the biopsy sample extraction device is transitionable to an armed position by removing at least one of the protective cap or the locking clip.

9. A biopsy sample extraction device comprising:
an elongated housing;
a sliding end coupled to a mating end of the elongated housing;
a compression-based actuation system including a rotatable blade coupled to a spring disposed in the elongated housing, the rotatable blade at least partially held in place by a trigger bar, and configured such that a force against a front face of the sliding end causes the trigger bar to release the rotatable blade; and
a dual-action locking system including one or more removable obstructions to prevent the trigger bar and the sliding end from moving.

10. The biopsy sample extraction device of claim 9,
wherein,
the one or more removable obstructions include at least a locking clip and a protective cap.

11. The biopsy sample extraction device of claim 10,
wherein,
the protective cap includes a locking post, extending from an inner surface of the protective cap, configured to occupy a space adjacent the trigger bar when the protective cap is disposed on the biopsy sample extraction device.

12. The biopsy sample extraction device of claim 11,
wherein,
the locking post includes a slanted end that faces inward with respect to a centerline of the elongated housing.

13. A biopsy extraction device of claim 12,
wherein,
the slanted end is a first slanted end; and
the sliding end includes one or more actuation members with one or more second slanted ends, the one or more second slanted ends facing outward with respect to the centerline of the elongated housing.

14. The biopsy sample extraction device of claim 9,
wherein,
the one or more removable obstructions include a locking clip having a first arm and a second arm.

15. The biopsy sample extraction device of claim 14,
wherein,
the first arm and the second arm are curved to correspond to a shape of the elongated housing.

16. The biopsy sample extraction device of claim 14,
wherein,
the locking clip includes a pull tab coupled the first arm and the second arm.

17. The biopsy sample extraction device of claim 16,
wherein,
the locking clip is disposed adjacent to a lip wrapping at least partially circumferentially around the elongated housing when the locking clip is preventing motion of the sliding end.

18. The biopsy sample extraction device of claim 9, further comprising:
one or more guide channels defined into a mating end of the elongated housing for slidably mating with the sliding end.

19. A method of forming a biopsy sample extraction device, the method comprising:
securing a rotatable blade in a locked position in a housing of the biopsy sample extraction device, the rotatable blade being coupled to a compressed spring disposed in the housing;
forming a slidable coupling between a sliding end and the housing;
positioning, as a first locking mechanism, a protective cap over at least a portion of the sliding end; and
positioning, as a second locking mechanism, a locking clip between the sliding end and the housing.

20. The method of claim 19, further comprising:
configuring the biopsy sample extraction device to create a biopsy sample with a rotational cut of the rotatable blade by:
configuring the protective cap to be pulled away by using a first force in a first direction;
configuring the locking clip to be pulled away by using a second force in a second direction; and
configuring the sliding end to receive a force which causes a living hinge to release a detent on the sliding end, thus releasing the compressed spring and causing the rotatable blade to rotate about a pin.
